(19) United States
(12) Patent Application Publication
(10) Pub. No.: US 2006/0106312 A1
(43) Pub. Date: May 18, 2006

(54) PEN DEVICE FOR MAPPING OF TISSUE

(76) Inventor: Richard C. Farmer, Peoria, AZ (US)

Correspondence Address: ELLIS & VENABLE, PC, 101 NORTH FIRST AVE., SUITE 1875, PHOENIX, AZ, 85003, US

(21) Appl. No.: 11/164,307

(22) Filed: Nov. 17, 2005

Related U.S. Application Data
(60) Provisional application No. 60/629,048, filed on Nov. 17, 2004.

(51) Int. Cl. A61B 8/14

(52) U.S. Cl. 600/459

(57) ABSTRACT

An transducer accessory or producing at least one guideline on a sonogram having a shadow mask adapted to an ultrasound transducer, and additionally useful for or marking skin using a marking device such as a pen.

This application claims the benefit of U.S. Provisional Patent Application: 60/629,048 filed on Nov. 17, 2004.

### FIELD OF THE INVENTION

The present invention relates to the field of marking skin during an ultrasound examination; for example, a method and pen device for mapping of human tissue on skin as an accessory for diagnostic ultrasound.

### BACKGROUND

Ultrasound is commonly used to help a surgeon in locating the structures of interest before an incision is made to save operation time. For example in vascular reconstruction, a sonographer typically may be used to determine the quality, diameter, and location of suitable veins and then mark the course on the patient's skin with a permanent marking pen.

In a typical vascular mapping procedure, the center of a transducer of a diagnostic ultrasound machine may be marked with a permanent marking pen line on the transducer (also referred to as an “ultrasound transducer”). Subsequently, the patient has ultrasound gel applied; for example, to the patient's leg, and the sonographer scans the leg to locate the vein and mark it. In order to mark the vein on the skin, the sonographer centers the vein in the middle of the ultrasound screen and then either indents the patient's skin in line with the marking on the center of the transducer or removes the ultrasound gel and marks the skin with a pen. Because the ultrasound procedure is usually done with the lights off, there is a potential to make a marking error. Other skin marking procedures using ultrasound have also been used such as straws and fingernails, but they all suffer from the same deficiencies.

Devices and methods that would help the sonographer correlate a sonogram to a transducer location would be valuable. Since the current methods have inherent inaccuracies associated with the procedures, a device and method that does not need to be aligned to the transducer of the ultrasound machine and that provides accurate and efficient delivery of a semi-permanent mark to the skin for localization of vessels of interest would be valuable for the physician to review the anatomical variations prior to any surgical procedure. The present invention increases the specificity and accuracy of the markings, increases the efficiency of the sonographer and decreases the complexity of the procedure leading to an increased acceptance by the patient and the sonographer (also known as a technician).

### SUMMARY

It is an object of the present invention to provide a transducer accessory for marking skin comprising: a shadow mask adapted to a transducer for producing at least one guide on a sonogram, and a base adapted to the transducer and comprising a pen actuator and a pen aligned with the shadow mask. Preferably, the transducer accessory further comprises a pen holder and the pen is removable. Preferably, the base is removable from the transducer, and the pen actuator is a mechanical actuator, a pneumatic actuator, an electro-mechanical actuator or combinations thereof. Preferably, the pen comprises a pen tip, a pen ampule and an ampule cap and ink; more preferably, the pen comprises a breakable plug or removable plug and most preferably, the pen comprises an ampule spring and non-clogging ink in the presence of ultrasound gel. In a preferred embodiment, the pen may be a ball point pen, a liquid dropper, a spray, a brush, a sponge or a label device. Further, the shadow mask may be preferably a film comprising at least two layers; more preferably, the film may be polyester layer and an adhesive layer with some of the adhesive layer absent to create the shadow on the sonogram. It is also preferable that the base comprises a back and further comprises an adhesive pad coupled to the back and to the ultrasound transducer, and the base comprises a lower end coupled to the shadow mask.

It is another object of the present invention to provide a transducer accessory for producing at least one guide on a sonogram comprising: a shadow mask coupled to a transducer. Preferably, the shadow mask is a film, imprinted material, layered material, transducer cover or combination thereof and may be circular, square, rectangle or any other shape or geometry that may aid in the identification of the target. It is also preferred that the shadow mask is a film comprises at least one layer, and more preferably the film comprises a polyester layer and an adhesive layer with some of the adhesive layer absent to create the shadow on the sonogram. It is also preferable that the transducer accessory further comprise a sterile bag having an inside and an outside wherein the shadow mask is coupled to the outside of the bag.

It is another object of the present invention to provide a transducer accessory for marking skin comprising: a shadow mask adapted to a transducer for producing at least one guide on a sonogram, a pen adapted to the transducer and aligned with the shadow mask, and a pen actuator to actuate the pen. Preferably, the pen is removable from the transducer and the pen actuator is a mechanical actuator, a pneumatic actuator, an electro-mechanical actuator or combinations thereof. Also preferably, the pen comprises a pen tip, a pen ampule and an ampule cap, ink, a breakable plug or removable plug, and an ampule spring and non-clogging ink in the presence of ultrasound gel. It is also preferable that the pen may be a ball point pen, a liquid dropper, a spray, a brush, a sponge or a label device. The shadow mask may preferably be a film having at least two layers such that the ultrasound waves, and more preferably, the film comprises a polyester layer and an adhesive layer with some of the adhesive layer absent to create the shadow on the sonogram.

It is yet an object of the present invention to provide a method of creating of a sonogram image comprising physically correlating a shadow mask with a physical location on a transducer and shadow masking an ultrasound signal to create a contrast on the sonogram image. Preferably, the present invention provides a method of physically correlating a pen with a contrast on a sonogram image; and more preferably actuating the pen to mark a tissue.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### BRIEF DESCRIPTION OF THE DRAWINGS

FIG. 1 shows an exploded view of one preferred embodiment of the invention.

FIG. 2 shows a perspective view of one preferred embodiment of the invention.

FIG. 3 shows an exploded view of one preferred embodiment of a pen.

FIG. 4 shows a top view of one preferred embodiment of a shadow mask

FIG. 5 shows a side view of one preferred embodiment of a shadow mask

FIG. 6 shows a side view of one preferred embodiment of a shadow mask and sterile bag.

FIG. 7 shows a perspective view of one preferred embodiment of the invention a shadow mask and sterile bag used with a transducer.

FIG. 8 shows a perspective view of one preferred embodiment of the invention used with a transducer.

### DESCRIPTION OF PREFERRED EMBODIMENTS

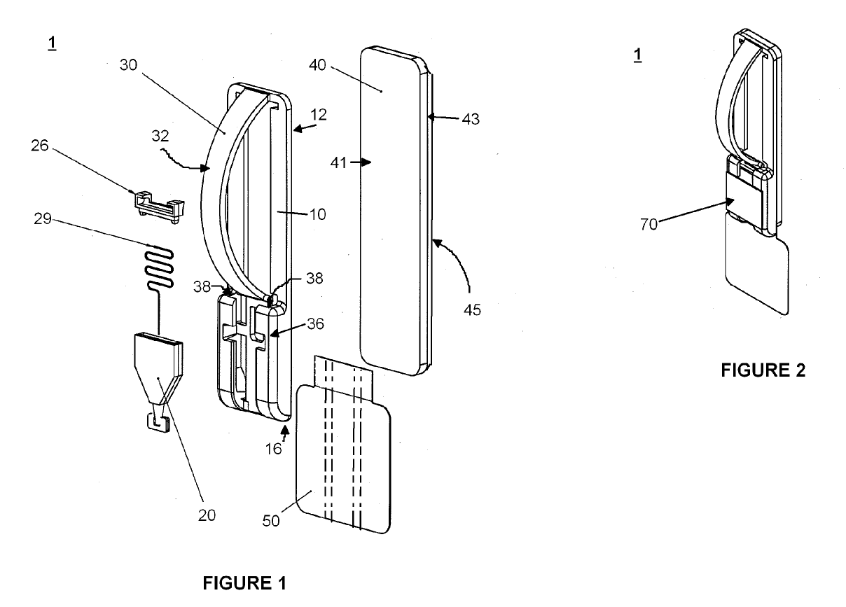

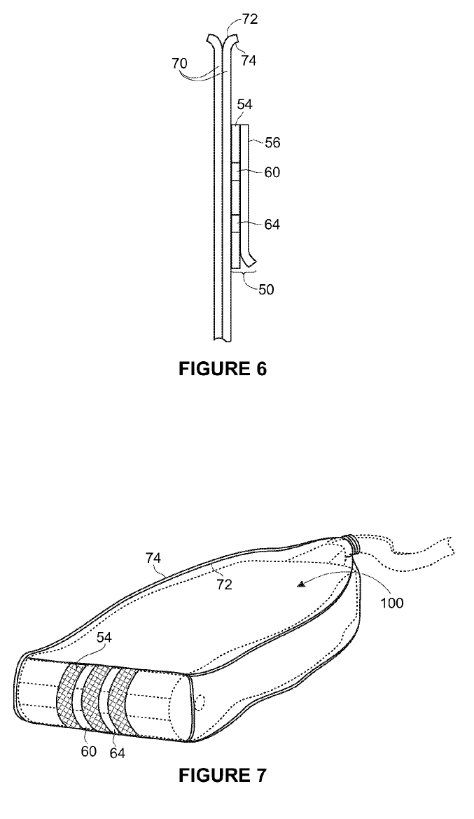

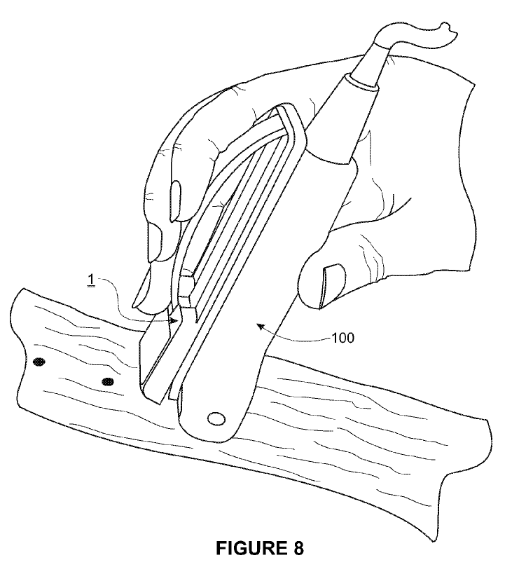

The present invention provides a method and device, also called an ultrasound accessory, for marking skin on a patient or animal. In one preferred embodiment of this invention shown in FIGS. 1-5 and 8, the device 1 comprises a base 10, a pen 20, a pen actuator 30, an adhesive pad 40, and a shadow mask 50. FIGS. 6 and 7 show another preferred embodiment of the invention.

As shown in the preferred embodiment in FIG. 1, the base 10 can be made of any material (or combination of materials) that does not interfere with operation of the ultrasound machine (not shown) and transducer 100. More preferably, the base 10 is made of plastic. In the preferred embodiment, the base 10 has a back 12 and a lower end 16.

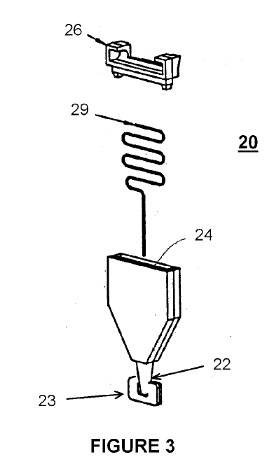

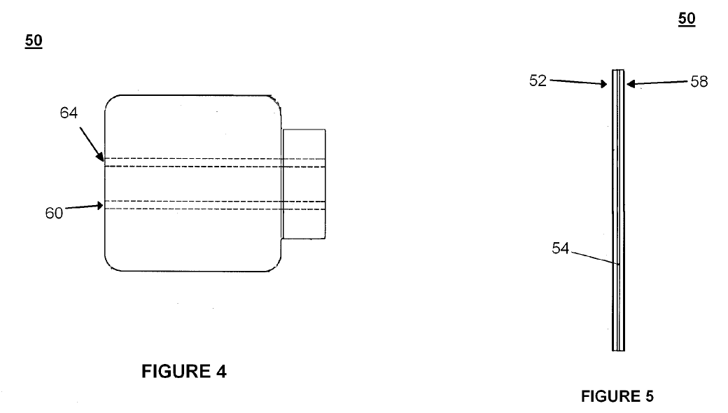

As shown in FIGS. 3 and 4, the preferred embodiment of the shadow mask 50 comprise a first layer 52, a second layer 54 and a mask release paper 56. While the shadow mask may comprise any combination of two or more materials that produce at least one guide, such as a shadow or contrast, on a sonogram, the preferred embodiment comprises a first layer 52 of polyester tape, and the second layer 54 comprises an adhesive layer. In the preferred embodiment shown in FIG. 3, a first groove 60 and second groove 64 are created in the adhesive layer. More preferably, the first groove 60 and second groove 64 are cut in the adhesive layer using a sharp edge and the material is removed. Alternatively, in another preferred embodiment, the groove or grooves may be created using separate strips of adhesive such that the separate strips of adhesive would bound the groove or grooves. Preferably the first grove and the second groove have widths between 0.5 and 3.5 mm. More preferably, both grooves have the same width, and most preferably the width of the grooves is about 1.78 mm (70 thousands of an inch). In the preferred embodiment of this invention, the distance between the first and the second groove needs to be enough to produce two shadow guidelines on a sonogram. In a more preferred embodiment, the width between the grooves is between 5 to 11 mm, and in the most preferred embodiment, the distance between the grooves is between 7 to 9 mm. In the preferred embodiment of this invention, the shadow mask 50 coupled to the lower end 16 of the base 10 such that the pen 20 is between the first groove 60 and second groove 64.

Preferably as shown in FIG. 1, the adhesive pad 40 is made of compliant foam and has adhesives on a first surface 41 and a second surface 43. In the preferred embodiment, the first surface 41 of the adhesive pad 40 is coupled to back 12 of the base 10, and the second surface 43 retains a pad release paper 45.

In the preferred embodiment of this invention, the pad release paper 45 is removed from the second surface 43 and the second surface 43 is attached to a side of a transducer 100 such that the shadow mask 50 may be pivoted over the working end of the transducer 100. More preferably, the mask release paper is removed from the adhesive second layer and the adhesive second layer is attached to the working surface of the transducer 100 as shown in FIG. 8. FIG. 8 illustrates the use of one preferred embodiment of the invention with a transducer in placing ink spots (black dots) on skin.

FIGS. 1 and 2 show the preferred embodiment of the pen 20 coupled to the base. In an additional preferred embodiment, a pen cover may used to hold the pen in place. More preferably, the pen 20 is aligned with the shadow mask and most preferably; the pen 20 is centered on the shadow mask. Preferably, the pen 20 includes an ink dispensing device. Additional embodiment of the invention include a pen of any type of dispensing devices such as fountain pens, ball point pens, liquid droppers, sprays, brushes, sponge, labels. Preferably, the pen may use any type of substance capable of marking skin such as ink, grease, paint, or other skin marking materials or chemicals and combinations thereof.

In a preferred embodiment of the invention as shown in FIG. 3, the pen comprises a pen tip 22, a pen ampule 24 and an ampule cap 26 and ink. More preferably, the pen tip has a plug 23 that may be broken to start the flow of ink through a small diameter hole in the pen tip 22. In a more preferred embodiment of the invention, the pen includes a spring 29 inside the pen ampule 24 such that it forces a small wire, spring or other ink drawing device through the small diameter hole in the pen tip 22. In this preferred embodiment, the pen actuator 30 causes the small wire, spring or other ink drawing device to contact the skin and be pushed into the pen ampule 24 (or pen tip 22) such that additional ink is released and/or ink is then moved forward along the wire, spring or other ink drawing device when the pen is no longer pushed into the pen ampule 24.

The ink may be any non-toxic ink that can be used with humans or animals. In the preferred embodiment of the invention, the ink is Gentian Violet (Surgical Marker Ink); more preferably, the Gentian violet ink is diluted with isopropyl alcohol or other suitable solution to keep the ultrasonic gel from clogging the pen tip 22. Another embodiment of invention includes any type of marking such as sterile label, fluorescent, luminous or reflective or any other kind of identification means that can be used to mark the target area.

The pen actuator 30 may be any mechanical, pneumatic or an electro-mechanical actuator or combinations thereof. In the preferred embodiment of FIG. 1, the pen actuator is a curved beam 32 such that it attaches to an upper end 14 of the base 10 and couples to the pen holder 36. In the preferred embodiment, pressing on the curved beam 32 caused the pen holder to slide towards the lower end 16 along the pen holder grooves 38 toward the area to be marked. Upon release of the curved beam 32, the elasticity of curved beam 32 causes the pen to return to its resting position. In an alternative embodiment; pen return springs may be placed at the front end of the pen to return the pen to its resting position.

An additional preferred embodiment of the invention includes a pen actuator that is built in or designed on the transducer where the pen is replaceable and disposable, and more preferably the pen is a one use only such that it is assembled with the unit before use and discarded after the marking procedure. In another preferred embodiment, the actuator and/or the pen could be coupled to the transducer in any manner such as adhesives, snap fit, holders or other fastening means. In an alternatively preferred embodiment, the pen tip would be centered above of the footprint of the sensing area of the transducer, having a shadow mask centered thereon, and the shadow mask would have a hole to allow the ink and/or the pen tip to contact the surface when activated by the pen actuator. This alternative embodiment would allow the sonographer to mark the target area directly rather than marking it in close proximity. This alternative embodiment may be actuated using any mechanical, pneumatic or electromechanical means.

In yet another alternative embodiment of the invention, the shadow mask may be imprinted on the footprint of the footprint of the transducer thereby allowing the sonographer to view use the invention without having the need to attach a film or material. The imprinted shadow mask may be any material such as silicon, vinyl, etc. and creates at least one area of different contrast on the sonogram. More preferably, shadow masks can be of any type of shape or geometry that may aid in the identification of the target such as circular, square, rectangle or any other shape; and most preferably, the shadow mask could made to conform to the particular shape of the transducer such as linear, curved linear, sector curve, etc.

In a yet another preferred embodiment of the invention, the shadow mask may be used as a stand alone transducer accessory. In a preferred embodiment used for a non-sterile application, a shadow mask could be attached directly to or imprinted on the transducer. In another preferred embodiment for sterile application as shown in FIGS. 6 and 7, an alternative shadow mask 50 having a second layer 54 and a mask release layer 56 may be attached to (or imprinted on) a sterile bag. Preferably, the sterile bag 70 has an outside 74 (not sterile) and an inside 72 (sterile) such that removing the second layer 54 may be attached to the outside layer 74 of the sterile bag 70. The sterile bag 70 may then be opened and turned inside out to cover the ultrasound transducer as shown in FIG. 7. Most preferably, the second layer 54 has adhesive on both sides.

In yet another embodiment, the shadow mask can be incorporated into a detachable transducer 100 cover that is adapted to the footprint of the sensing area of the transducer 100. More preferably, the transducer cover may either be disposable or reusable.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A transducer accessory for marking skin comprising: a shadow mask adapted to a transducer for producing at least one guide on a sonogram, and a base adapted to the transducer and comprising a pen actuator and a pen aligned with the shadow mask.

2. The transducer accessory in claim 1 wherein the base further comprises a pen holder and the pen is removable.

3. The transducer accessory in claim 2 wherein the base is removable from the transducer.

4. The transducer accessory in claim 1 wherein the pen actuator is a mechanical actuator, a pneumatic actuator, an electro-mechanical actuator or combinations thereof.

5. The transducer accessory in claim 1 wherein the pen comprises a pen tip, a pen ampule and an ampule cap and ink.

6. The transducer accessory in claim 5 wherein the pen further comprises a breakable plug or removable plug.

7. The transducer accessory in claim 6 wherein the pen further comprises an ampule spring and non-clogging ink in the presence of ultrasound gel.

8. The transducer accessory in claim 1 wherein the pen is a ball point pen, a liquid dropper, a spray, a brush, a sponge or a label device.

9. The transducer accessory in claim 1 wherein the shadow mask is a film comprising at least two layers.

10. The transducer accessory in claim 1 wherein the shadow mask comprises a polyester layer and an adhesive layer with some of the adhesive layer absent to create the shadow on the sonogram.

11. The transducer accessory in claim 1 wherein the base comprises a back and further comprising an adhesive pad coupled to the back and to the transducer.

12. The transducer accessory in claim 1 wherein the base comprises a lower end coupled to the shadow mask.

13. A transducer accessory for producing at least one guide on a sonogram comprising: a shadow mask coupled to a transducer.

14. The transducer accessory in claim 13 wherein the shadow mask is a film, imprinted material, layered material, transducer cover or combination thereof.

15. The transducer accessory in claim 13 wherein the shadow mask is circular, square, rectangle or any other shape or geometry that may aid in the identification of the target.

16. The transducer accessory in claim 14 wherein the shadow mask is a film comprising at least one layer.

17. The transducer accessory in claim 16 wherein the shadow mask comprises a polyester layer and an adhesive layer with some of the adhesive layer absent to create the shadow on the sonogram.

18. The transducer accessory in claim 13 further comprising a sterile bag having an inside and an outside wherein the shadow mask is coupled to the outside of the bag.

19. A transducer accessory for marking skin comprising: a shadow mask adapted to a transducer for producing at least one guide on a sonogram, a pen adapted to the transducer and aligned with the shadow mask, and a pen actuator to actuate the pen.

20. The transducer accessory in claim 19 wherein the pen is removable from the transducer.

21. The transducer accessory in claim 19 wherein the pen actuator is a mechanical actuator, a pneumatic actuator, an electro-mechanical actuator or combinations thereof.

22. The transducer accessory in claim 22 wherein the pen comprises a pen tip, a pen ampule and an ampule cap and ink.

23. The transducer accessory in claim 22 wherein the pen ampule is removable.

24. The transducer accessory in claim 22 wherein the pen further comprises a breakable plug or removable plug.

25. The transducer accessory in claim 24 wherein the pen further comprises an ampule spring and non-clogging ink in the presence of ultrasound gel.

26. The transducer accessory in claim 19 wherein the pen is a ball point pen, a liquid dropper, a spray, a brush, a sponge or a label device.

27. The transducer accessory in claim 19 wherein the shadow mask is a film comprising at least two layers such that the ultrasound waves produce a shadow on the sonogram.

28. The transducer accessory in claim 19 wherein the shadow mask comprises a polyester layer and an adhesive layer with some of the adhesive layer absent to create the shadow on the sonogram.

29. The transducer accessory in claim 19 wherein the transducer has a working surface center and the shadow mask is centered thereon, and wherein the pen is adapted to the working surface center and the shadow mark to enable marking directly over the working surface center.

30. The transducer accessory in claim 29 wherein the working surface center and the shadow mask have at least one hole to allow the pen to protrude through.

31. A method of creating of a sonogram image comprising physically correlating a shadow mask with a physical location on a transducer and shadow masking an ultrasound signal to create a contrast on the sonogram image.
